(19) United States
(12) Patent Application Publication
(10) Pub. No.: US 2026/0157993 A1
(43) Pub. Date: Jun. 11, 2026

(54) USE OF 2-METHYLOXOLANE FOR FEEDING OVIPAROUS ANIMALS

(71) Applicant: ECOXTRACT, DUNKERQUE (FR)

(72) Inventors: Mickael BARTIER, DUNKERQUE (FR); Laurence JACQUES, DUNKERQUE (FR)

(73) Assignee: ECOXTRACT, DUNKERQUE (FR)

(21) Appl. No.: 19/118,570

(22) PCT Filed: Oct. 5, 2023
(86) PCT No.: PCT/FR2023/051552

§ 371 Date: Apr. 4, 2025

(30) Foreign Application Priority Data
Oct. 6, 2022 (FR) FR2210258

(51) Int. Cl. A61K 31/341; A23K 10/30; A23K 20/121; A23K 50/75; A61K 9/00; A61K 47/46

(52) U.S. Cl. CPC A61K 31/341; A23K 10/30; A23K 20/121; A23K 50/75; A61K 9/0053; A61K 47/46

(57) ABSTRACT

A method for increasing the egg production of an oviparous animal. The method includes the administration of a food including 2-methyloxolane and a composition for feeding an oviparous animal. Also, a dietary supplement, such as in the form of a biological substrate cake, for feeding an oviparous animal including 2-methyloxolane, and a kit including the dietary supplement.

### FIELD

The present disclosure relates to the field of feeding oviparous animals, in particular gallinaceans, more particularly chickens.

### BACKGROUND

Oleaginous plant cakes are commonly used in animal feed as a protein supplement, but also as a metabolisable energy source. Oleaginous plant cakes are the solid residues derived from the process of food oil production by solid/liquid extraction of oleaginous plant grains.

Currently, hexane is the most used solvent in the solid/liquid extraction process because it has advantageous properties. Indeed, hexane allows for a high-efficiency extraction of oil (>97%) and allows leaving less than 3% residual oil in the solid substrate, thereby avoiding the latter becoming rancid. Hexane is easy to separate from the extracted oil, it has a suitable boiling point (i.e. high enough to limit losses during extraction but low enough to limit energy consumption during separation thereof with the extracted oil and during recycling thereof), it is stable and has a good lipid selectivity.

Oil cakes comprise hexane and their hexane content is regulated in some territories. For example, in Europe, the acceptable maximum hexane content in the cakes is 1,000 mg/kg.

Nevertheless, hexane has major drawbacks. It is derived from petroleum, it is neurotoxic and is classified as toxic for category 2 reproduction and as toxic for category 2 aquatic environment. It is classified in the “solvent to be limited” category by the pharmaceutical industry experts (ICHQ3C R8) with an Acceptable Daily Dose for an average human of 50 kg of only 2.9 mg.

Hence, hexane alternative solvents have been tested on a laboratory and pilot scale. For example, application EP 19 842 832.3 by the Applicant describes the production of defatted cakes by solid/liquid extraction of soybean, rapeseed, corn, cotton and sunflower, with a binary mixture of 2-methyloxolane and water.

Seeking to quantify the presence of 2-methyloxolane residues in chicken eggs, the Applicant has observed that, quite unexpectedly, the chickens whose feed is supplemented with 2-methyloxolane would lay more eggs than the chickens whose feed is not supplemented with 2-methyloxolane.

### SUMMARY

Thus, there is provided a use of a dietary supplement comprising 2-methyloxolane to increase the egg production of an oviparous animal, in particular a gallinacean, more particularly a chicken.

Advantageously, the oviparous animal whose feed is supplemented by the dietary supplement comprising 2-methyloxolane lays up to 14% more eggs per week than the oviparous animal whose feed is not supplemented by the dietary supplement according to the invention.

2-methyloxolane is also non-toxic when ingested for the amounts considered in animal feed. Indeed, a published test of 3-month ingestion on rats has showed a dose with no undesirable effect observed for 250 mg/kg body weight per day.

Thus, the dietary supplement comprising 2-methyloxolane is not toxic to animals whose feed is supplemented by said dietary supplement.

Moreover, the concentration of 2-methyloxolane residues present in the meat originating from the oviparous animal whose feed is supplemented by the dietary supplement comprising 2-methyloxolane and in the eggs laid by the oviparous animal whose feed is supplemented by the dietary supplement comprising 2-methyloxolane is very low and compatible with the human exposure limit recommended by EFSA of 1 mg/kg human body weight per day (EFSA Opinion on 2-methyloxolane of Mar. 15, 2022).

In addition, some oviparous animals whose feed is supplemented by the dietary supplement comprising 2-methyloxolane can have their weight significantly increase.

Furthermore, the 2-methyloxolane is of bio-sourced origin because its raw material is typically derived from sugar cane bagasse or corncobs. In addition, it is not classified as toxic to the environment.

According to another aspect, there is provided a dietary supplement for feeding an oviparous animal, in particular a gallinacean, more particularly a chicken, said dietary supplement comprising 2-methyloxolane.

According to another aspect, there is provided the use of a kit for increasing the egg production of an oviparous animal, in particular a gallinacean, more particularly a chicken, said kit comprising:
the dietary supplement comprising 2-methyloxolane as described hereinabove, and
a composition for feeding an oviparous animal, in particular a gallinacean, more particularly a chicken.

According to another aspect, there is provided a kit for feeding an oviparous animal, in particular a gallinacean, more particularly a chicken, said kit comprising:
the dietary supplement comprising 2-methyloxolane as described hereinabove, and
a composition for feeding an oviparous animal, in particular a gallinacean, more particularly a chicken.

According to another aspect, there is provided the use of a food for increasing the egg production of an oviparous animal, in particular a gallinacean, more particularly a chicken, said food comprising the dietary supplement comprising 2-methyloxolane as described hereinabove and a composition for feeding an oviparous animal, in particular a gallinacean, more particularly a chicken as described hereinabove.

According to another aspect, there is provided a food intended for feeding an oviparous animal, in particular a gallinacean, more particularly a chicken, said food comprising the dietary supplement comprising 2-methyloxolane as described hereinabove and a composition for feeding an oviparous animal, in particular a gallinacean, more particularly a chicken.

According to another aspect, there is provided a dietary supplement as described hereinabove, a kit as described hereinabove or a food as described hereinabove for use thereof in increasing the egg production of an oviparous animal, in particular a gallinacean, more particularly a chicken.

According to another aspect, there is provided a method for increasing the egg production of an oviparous animal, in particular a gallinacean, more particularly a chicken, comprising a step of ingestion by the oviparous animal, in particular the gallinacean, more particularly the chicken, of the dietary supplement as described hereinabove, of the kit as described hereinabove or of the food as described hereinabove.

### DETAILED DESCRIPTION

According to a first aspect, there is provided a use of a dietary supplement comprising 2-methyloxolane to increase the egg production of an oviparous animal, in particular a gallinacean, more particularly a chicken.

In the context of the present application, “oviparous animal” refers to an animal that lays eggs and whose embryo growth takes place in these. A bird of the gallinaceans family is an example of an oviparous animal.

In the context of the present application, “gallinacean” refers to a family of omnivorous birds having as common characteristics a stocky body, a thick spout, short and rounded wings, robust legs. The gallinaceans family comprises several sub-families. Phasianidae, including chickens, cocks, quails, peacocks, partridges, and guinea fowls is one of these subfamilies.

In the context of the present application, “dietary supplement” refers to a foodstuff intended to complete a normal diet and which forms a concentrated source of nutrients or other substances having a nutritional or physiological effect.

Typically, the amount of 2-methyloxolane administered to the oviparous animal, in particular to a gallinacean, more particularly to a chicken, in this use may be comprised between 0.1 mg/kg of the oviparous animal per day and 200 mg/kg of the oviparous animal per day, in particular between 1 mg/kg of the oviparous animal per day and 50 mg/kg of the oviparous animal per day, more particularly between 3 mg/kg of the oviparous animal per day and 32 mg/kg of the oviparous animal per day.

Advantageously, the egg production of the oviparous animal increases as the amount of 2-methyloxolane ingested by said oviparous animal is dosed within these ranges.

The used dietary supplement may be in the form of a solution, a powder, a capsule, an extrudate, a tablet or a biological substrate cake, in particular in the form of a powder, a capsule or a biological substrate cake, more particularly a biological substrate cake.

In the used dietary supplement, the 2-methyloxolane may be in the liquid form or be impregnated over a food-grade carrier for the oviparous animal of the used dietary supplement.

For example, the 2-methyloxolane in the liquid form is adapted to the dietary supplement in the solution and capsule form. The impregnation of 2-methyloxolane is adapted to the dietary supplement in the form of a powder, an extrudate, a tablet or a biological substrate cake.

These formats are easy to produce because they are known to a person skilled in the art and 2-methyloxolane is liquid under atmospheric pressure and temperature conditions. Hence, it can be easily impregnated by known techniques on the food-grade carrier.

For example, the food-grade carrier of the dietary supplement may be selected from among a mineral or biological carrier. For example, the mineral carrier may be a mineral powder, such as a silica powder. The Tixosil® 68 powder is an example of a silica powder adapted to be used as a mineral carrier.

In the context of the present application, by “biological substrate cake”, it should be understood the solid residue of the extraction of oil or juice from a biological substrate.

In the context of the present application, by “biological substrate”, it should be understood a solid material selected from among a plant, an alga, a microorganism and mixtures thereof, in particular a plant.

For example, the plant may be an oleaginous plant, a proteaginous plant, or a mixture thereof.

As oleaginous plants, mention may be made of rapeseed, sunflower, soybean, peanut, sesame, walnut, almond, cotton, flax or mixtures thereof, in particular rapeseed, sunflower, soybean or mixtures thereof.

Examples of proteaginous plants include peas, beans, lupin beans and mixtures thereof.

Typically, the biological substrate may be a plant selected from among almond, amaranth, peanut, argan, sea buckthorn, cashew tree, avocado, oats, borage, safflower, camelina, carrot, cocao, cashew, canola, hemp, rapeseed, copra, marrow, cotton, croton, rosehip, fig, prickly pear, pomegranate, hop, illipe, jojoba, jasmine, shea tree, lavender, lavandin, flax, lupin bean, corn, hazel tree, walnut, coconut palm, poppy, olive, evening-primrose, orange tree, oil palm, paprika, pecan, pistachio, pepper, castor, rice, rose, cedar rose, musk rose, sesame, soybean, marigold, sunflower, Calophyllum inophyllum, madhuca, Queensland nut, raspberry, blackcurrant, melon, grape, tomato, baobab, babassu, cranberry, chia, pumpkin, mustard, neem, Nigella Sativa, niger, poppy, Perilla, Plukenetia volubili winter squash, annatto, Taramira, apricot, plum, peach, wheat, tuberose, ylang-ylang, geranium, patchouli, petitgrain (citrus), anise, caraway, juniper, bergamot, mandarin orange, grapefruit, lemon, nutmeg tree, celery, angelica, cardamom, cumaru, vanilla, vetiver, iris, sandalwood, guaiacum, tarragon, citronella, sage, mint, Pine tree, rosemary, fir tree, thyme, galbanum, elemi, benzoin, myrrh, olibanum tree and mixtures thereof, in particular soybean, rapeseed, sunflower, baobab, corn, peanut, coconut palm, oil palm, cotton and mixtures thereof, quite particularly soybean, rapeseed, sunflower, baobab and mixtures thereof.

According to a specific embodiment, the biological substrate cake is a cake of soybean, rapeseed, sunflower, corn, peanut and mixtures thereof.

The biological substrate cake may be in the form of cake or extrudate flour.

In the context of the present application, by “cake flour”, it should be understood the by-product derived from grinding, milling or spraying of the cake in order to obtain a powder thereof.

Advantageously, the cake flour is easy to eat and is very well assimilated by an oviparous animal.

In the context of the present application, by “extrudate”, it should be understood a product whose production is done by extrusion.

The biological substrate cake may be derived from a process for producing oil by solid/liquid extraction using 2-methyloxolane. Such a method is described, for example, in application EP 19 842 832.3.

Typically, the mass concentration of 2-methyloxolane in the biological substrate cake may be lower than 1,000 mg/kg, in particular from 10 mg/kg to 500 mg/kg, quite particularly from 100 mg/kg to 300 mg/kg.

Advantageously, a mass concentration within these ranges allows administering an effective dose of 2-methyloxolane to the animal while ingesting an acceptable amount of biological substrate cake.

According to another aspect, there is provided a dietary supplement for feeding an oviparous animal, in particular a gallinacean, more particularly a chicken, said dietary supplement comprising 2-methyloxolane.

The dietary supplement may be in the form of a solution, a powder, a capsule, an extrudate, a tablet or a biological substrate cake, in particular a powder, a capsule or a biological substrate cake, more particularly a biological substrate cake.

In the dietary supplement, the 2-methyloxolane may be in the liquid form or be impregnated on a food-grade carrier for the oviparous animal of the dietary supplement.

For example, the 2-methyloxolane in the liquid form is adapted to the dietary supplement in the solution and capsule form. The impregnation of 2-methyloxolane is adapted to the dietary supplement in the form of a powder, an extrudate, a tablet or a biological substrate cake.

These formats are easy to produce because they are known to a person skilled in the art and 2-methyloxolane is liquid under atmospheric pressure and temperature conditions. Hence, it can be easily impregnated by known techniques on the food-grade carrier.

For example, the food-grade carrier of the dietary supplement may be selected from among a mineral or biological carrier. For example, the mineral carrier may be a mineral powder, such as a silica powder. The Tixosil® 68 powder is an example of a silica powder adapted to be used as a mineral carrier.

As defined hereinabove, the biological substrate cake is the solid residue of the extraction of oil or juice from a biological substrate which is a solid material selected from among a plant, an alga, a microorganism and mixtures thereof, in particular a plant.

For example, the plant may be an oleaginous plant, a proteaginous plant, or a mixture thereof.

As oleaginous plants, mention may be made of rapeseed, sunflower, soybean, peanut, sesame, walnut, almond, cotton, flax or mixtures thereof, in particular rapeseed, sunflower, soybean or mixtures thereof.

Examples of proteaginous plants include peas, beans, lupin beans and mixtures thereof.

Typically, the biological substrate may be a plant selected from among almond, amaranth, peanut, argan, sea buckthorn, cashew tree, avocado, oats, borage, safflower, camelina, carrot, cocao, cashew, canola, hemp, rapeseed, copra, marrow, cotton, croton, rosehip, fig, prickly pear, pomegranate, hop, illipe, jojoba, jasmine, shea tree, lavender, lavandin, flax, lupin bean, corn, hazel tree, walnut, coconut palm, poppy, olive, evening-primrose, orange tree, oil palm, paprika, pecan, pistachio, pepper, castor, rice, rose, cedar rose, musk rose, sesame, soybean, marigold, sunflower, Calophyllum inophyllum, madhuca, Queensland nut, raspberry, blackcurrant, melon, grape, tomato, baobab, babassu, cranberry, chia, pumpkin, mustard, neem, Nigella Sativa, niger, poppy, Perilla, Plukenetia volubili winter squash, annatto, Taramira, apricot, plum, peach, wheat, tuberose, ylang-ylang, geranium, patchouli, petitgrain (citrus), anise, caraway, juniper, bergamot, mandarin orange, grapefruit, lemon, nutmeg tree, celery, angelica, cardamom, cumaru, vanilla, vetiver, iris, sandalwood, guaiacum, tarragon, citronella, sage, mint, Pine tree, rosemary, fir tree, thyme, galbanum, elemi, benzoin, myrrh, olibanum tree and mixtures thereof, in particular soybean, rapeseed, sunflower, baobab, corn, peanut, coconut palm, oil palm, cotton and mixtures thereof, quite particularly soybean, rapeseed, sunflower, baobab and mixtures thereof.

According to a specific embodiment, the biological substrate cake is a cake of soybean, rapeseed, sunflower, corn, peanut and mixtures thereof.

The biological substrate cake may be in the form of a cake or extrudate flour.

Advantageously, the cake flour is easy to eat and is very well assimilated by an oviparous animal.

The biological substrate cake may be derived from a process for producing oil by solid/liquid extraction using 2-methyloxolane. Such a process is described, for example, in application EP 19 842 832.3.

Typically, the mass concentration of 2-methyloxolane in the biological substrate cake may be lower than 1,000 mg/kg, in particular from 10 mg/kg to 500 mg/kg, quite particularly from 100 mg/kg to 300 mg/kg.

Advantageously, a mass concentration within these ranges allows administering an effective dose of 2-methyloxolane to the animal while making it ingest an acceptable amount of biological substrate cake.

The dietary supplement may be ingested by the oviparous animal as a supplement to its conventional feed, for example as a supplement to grains.

Thus, according to another aspect, there is provided the use of a kit for increasing the egg production of an oviparous animal, in particular a gallinacean, more particularly a chicken, said kit comprising:
the dietary supplement comprising 2-methyloxolane as described hereinabove, and
a composition for feeding an oviparous animal, in particular a gallinacean, more particularly a chicken.

Typically, the amount of 2-methyloxolane administered to the oviparous animal, in particular a gallinacean, more particularly a chicken, when using the kit may be comprised between 0.1 mg/kg of the oviparous animal per day and 200 mg/kg of the oviparous animal per day, in particular between 1 mg/kg of the oviparous animal per day and 50 mg/kg of the oviparous animal per day, more particularly between 3 mg/kg of the oviparous animal per day and 32 mg/kg of the oviparous animal per day.

Advantageously, the egg production of the oviparous animal increases as the amount of 2-methyloxolane ingested by said oviparous animal is dosed within these ranges.

For example, the composition for feeding an oviparous animal of the used kit may be:
a cereal grain like wheat, corn, oats, and mixtures thereof,
a food waste like fruit and/or vegetable peelings, and/or
a complete food,

in particular a complete food.

In the context of the present application, “complete food” refers to a compound food, suitable from the nutritional perspective to the oviparous animals thanks to a specific composition, and designed to form a unique ration that could keep the oviparous animals alive and/or promote egg production.

The complete food may be in the form of an extrudate, a granulate, a powder or a pellet.

According to another aspect, there is provided a kit for feeding an oviparous animal, in particular a gallinacean, more particularly a chicken, said kit comprising:
the dietary supplement comprising 2-methyloxolane as described hereinabove, and
a composition for feeding an oviparous animal, in particular a gallinacean, more particularly a chicken.

For example, the composition for feeding animals of the kit may be:
a cereal seed like wheat, corn, oats, and mixtures thereof,
a food waste like fruit and/or vegetable peelings, and/or
a complete food,

in particular a complete food.

The complete food may be in the form of an extrudate, a granulate, a powder or a pellet.

The dietary supplement and the composition for feeding animals, in particular the complete feed, may form a food intended for feeding an oviparous animal, in particular a gallinacean, more particularly a chicken.

Thus, according to another aspect, there is provided the use of a food to increase the egg production of an oviparous animal, in particular a gallinacean, more particularly a chicken, said food comprising the dietary supplement comprising 2-methyloxolane as described hereinabove and a composition for feeding an oviparous animal, in particular a gallinacean, more particularly a chicken, as described hereinabove.

In the context of the present application, “food” refers to an appropriate composition from the nutritional perspective to the oviparous animals thanks to its specific composition, and designed to form a unique ration that could keep the oviparous animals alive and/or promote egg production.

Advantageously, the used food is easy to implement by the breeder as it allows feeding, with a unique ration, the oviparous animal in order to increase its egg production.

Typically, the amount of 2-methyloxolane administered to the oviparous animal, in particular a gallinacean, more particularly a chicken, when using this food may be comprised between 0.1 mg/kg of the oviparous animal per day and 200 mg/kg of the oviparous animal per day, in particular between 1 mg/kg of the oviparous animal per day and 50 mg/kg of the oviparous animal per day, more particularly between 3 mg/kg of the oviparous animal per day and 32 mg/kg of the oviparous animal per day.

Advantageously, the egg production of the oviparous animal increases as the amount of 2-methyloxolane ingested by said oviparous animal is dosed within these ranges.

According to another aspect, there is provided a food for feeding an oviparous animal, in particular a gallinacean, more particularly a chicken, said food comprising the dietary supplement comprising 2-methyloxolane as described hereinabove and a composition for feeding an oviparous animal, in particular a gallinacean, more particularly a chicken as described hereinabove.

The food may be in the form of an extrudate, a granulate, a powder, or a pellet.

The food may be produced thanks to the method comprising the following steps of:
a1) mixing the dietary supplement and the composition for feeding animals to obtain a mixture,
b1) shaping the mixture obtained in step a) to produce the food.

Steps a1) and b1) are conventional steps that can be easily implemented by a person skilled in the art.

This production method is adapted to the food in the form of an extrudate, a granulate, a powder or a pellet.

Alternatively, the food may be produced thanks to the method comprising the following steps of:
a2) grinding the dietary supplement to obtain a first powder,
b2) grinding the composition for feeding animals to obtain a second powder,
c2) mixing the first powder and the second powder to produce the food.

Steps a2), b2) and c2) are conventional steps that can be easily implemented by a person skilled in the art.

This alternative production method is adapted to the food in the form of powder.

According to another aspect, there is provided a dietary supplement as described hereinabove, a kit as described hereinabove or a food as described hereinabove for use thereof in increasing the egg production of an oviparous animal, in particular a gallinacean, more particularly a chicken.

Typically, the amount of 2-methyloxolane administered to the oviparous animal, in particular a gallinacean, more particularly a chicken, thanks to the dietary supplement, to the kit or to the food for this use may be comprised between 0.1 mg/kg of the oviparous animal per day and 200 mg/kg of the oviparous animal per day, in particular between 1 mg/kg of the oviparous animal per day and 50 mg/kg of the oviparous animal per day, more particularly between 3 mg/kg of the oviparous animal per day and 32 kg of the oviparous animal per day.

Advantageously, the egg production of the oviparous animal increases as the amount of 2-methyloxolane ingested by said oviparous animal is dosed within these ranges.

According to another aspect, there is provided a method for increasing the egg production of an oviparous animal, in particular a gallinacean, more particularly a chicken comprising a step of ingestion by the oviparous animal, in particular the gallinacean, more particularly the chicken, of the dietary supplement as described hereinabove, of the kit as described hereinabove or of the food as described hereinabove.

Typically, during this method, the amount of 2-methyloxolane administered to the oviparous animal, in particular a gallinacean, more particularly a chicken, may be comprised between 0.1 mg/kg of the oviparous animal per day and 200 mg/kg of the oviparous animal per day, in particular between 1 mg/kg of the oviparous animal per day and 50 mg/kg of the oviparous animal per day, more particularly between 3 mg/kg of the oviparous animal per day and 32 mg/kg of the oviparous animal per day.

Advantageously, the egg production of the oviparous animal increases as the amount of 2-methyloxolane ingested by said oviparous animal is dosed within these ranges.

### Examples

The following examples allow illustrating the invention yet without limiting it.

In this example, domestic laying hens have been divided into four groups:
the 4 chickens of the control group have not ingested 2-methyloxolane.
the 12 chickens of the N×1 group have ingested pure liquid 2-methyloxolane in capsules. The ingested dose of pure liquid 2-methyloxoane was 3.18 mg/kg of the chicken/day, i.e. 40 mg/kg in the daily ration,
the 12 chickens of the N×5 group have ingested pure liquid 2-methyloxolane in capsules. The ingested dose of pure liquid 2-methyloxoane was 15.88 mg/kg of the chicken/day, i.e. 200 mg/kg in the daily ration, and
the 12 chickens of the N×10 group have ingested pure liquid 2-methyloxolane in capsules. The ingested dose of pure liquid 2-methyloxoane was 31.77 mg/kg of the chicken/day, i.e. 400 mg/kg in the daily ration (the group N×10).

The egg production of each group has been monitored in order to statistically validate the significance of the effect of an oral administration of 2-methyloxolane on egg production.

The statistical processing is as follows:

A one-factor analysis of variance (ANOVA) has been carried out using the XLSTAT 2020.2.1 software on MS EXCEL 16.0.15225. The significance threshold is 95% with a p value of 0.05 to verify the significant effect of an oral administration of 2-methyloxolane on egg production, with:
the discrete explanatory variable: basking group and
the continuous variable to be explained: number of laid eggs.

In case of a significant difference between the averages, a Student's t-test with a level of significance of 5% will be applied for the comparison in pairs using the same software.

In total, 1,115 eggs have been counted throughout the observations.

Table 1 hereinbelow presents the average of the number of eggs per day laid by 4 chickens as a function of their basking group.

TABLE 1

| Basking group | Number of eggs |
|---|---|
| Control | 3.5 |
| N × 1 | 3.9 |
| N × 5 | 3.9 |
| N × 10 | 3.9 |

The Student's t-test has clearly demonstrated the existence of 2 groups:
the control group, and
groups that have received 2-methyloxolane orally.

Hence, this example demonstrates that an oral administration of 2-methyloxolane significantly increases the number of laid eggs per chicken without any apparent dose effect in the tested range, namely from 40 to 400 mg/kg of 2-methyloxolane in daily ration.

1-13. (canceled)

14. A method for increasing the egg production of an oviparous animal, comprising the administration to the oviparous animal of a dietary supplement comprising 2-methyloxolane.

15. The method according to claim 14, wherein the amount of 2-methyloxolane administered to the oviparous animal is comprised between 0.1 mg/kg of the oviparous animal per day and 200 mg/kg of the oviparous animal per day.

16. The method according to claim 14, wherein the dietary supplement is in the form of a solution, a powder, a capsule, an extrudate, a tablet or is a biological substrate cake.

17. The method according to claim 14, wherein the dietary supplement is a biological substrate cake.

18. The method according to claim 17, wherein the biological substrate cake is in the form of a cake or extrudate flour.

19. The method according to claim 17, wherein the biological substrate may be a plant selected from among almond, amaranth, peanut, argan, sea buckthorn, cashew tree, avocado, oats, borage, safflower, camelina, carrot, cocao, cashew, canola, hemp, rapeseed, copra, marrow, cotton, croton, rosehip, fig, prickly pear, pomegranate, hop, illipe, jojoba, jasmine, shea tree, lavender, lavandin, flax, lupin bean, corn, hazel tree, walnut, coconut palm, poppy, olive, evening-primrose, orange tree, oil palm, paprika, pecan, pistachio, pepper, castor, rice, rose, cedar rose, musk rose, sesame, soybean, marigold, sunflower, Calophyllum inophyllum, madhuca, Queensland nut, raspberry, blackcurrant, melon, grape, tomato, baobab, babassu, cranberry, chia, pumpkin, mustard, neem, Nigella Sativa, niger, poppy, Perilla, Plukenetia volubili winter squash, annatto, Taramira, apricot, plum, peach, wheat, tuberose, ylang-ylang, geranium, patchouli, petitgrain (citrus), anise, caraway, juniper, bergamot, mandarin orange, grapefruit, lemon, nutmeg tree, celery, angelica, cardamom, cumaru, vanilla, vetiver, iris, sandalwood, guaiacum, tarragon, citronella, sage, mint, Pine tree, rosemary, fir tree, thyme, galbanum, elemi, benzoin, myrrh, olibanum tree and mixtures thereof.

20. A method for increasing the egg production of an oviparous animal comprising the administration to the oviparous animal of a kit, said kit comprising:
a dietary supplement comprising 2-methyloxolane, and
a composition for feeding an oviparous animal.

21. The method according to claim 20, wherein the amount of 2-methyloxolane administered to the oviparous animal is comprised between 0.1 mg/kg of the oviparous animal per day and 200 mg/kg of the oviparous animal per day.

22. The method according to claim 20, wherein the dietary supplement is in the form of a solution, a powder, a capsule, an extrudate, a tablet or is a biological substrate cake.

23. The method according to claim 22, wherein the dietary supplement is a biological substrate cake.

24. The method according to claim 22, wherein the biological substrate cake is in the form of a cake or extrudate flour.

25. The method according to claim 22, wherein the biological substrate may be a plant selected from among almond, amaranth, peanut, argan, sea buckthorn, cashew tree, avocado, oats, borage, safflower, camelina, carrot, cocao, cashew, canola, hemp, rapeseed, copra, marrow, cotton, croton, rosehip, fig, prickly pear, pomegranate, hop, illipe, jojoba, jasmine, shea tree, lavender, lavandin, flax, lupin bean, corn, hazel tree, walnut, coconut palm, poppy, olive, evening-primrose, orange tree, oil palm, paprika, pecan, pistachio, pepper, castor, rice, rose, cedar rose, musk rose, sesame, soybean, marigold, sunflower, Calophyllum inophyllum, madhuca, Queensland nut, raspberry, blackcurrant, melon, grape, tomato, baobab, babassu, cranberry, chia, pumpkin, mustard, neem, Nigella Sativa, niger, poppy, Perilla, Plukenetia volubili winter squash, annatto, Taramira, apricot, plum, peach, wheat, tuberose, ylang-ylang, geranium, patchouli, petitgrain (citrus), anise, caraway, juniper, bergamot, mandarin orange, grapefruit, lemon, nutmeg tree, celery, angelica, cardamom, cumaru, vanilla, vetiver, iris, sandalwood, guaiacum, tarragon, citronella, sage, mint, Pine tree, rosemary, fir tree, thyme, galbanum, elemi, benzoin, myrrh, olibanum tree and mixtures thereof.

26. A method for increasing the egg production of an oviparous animal, comprising the administration of a food comprising a dietary supplement comprising 2-methyloxolane and a composition for feeding an oviparous animal.

27. The method according to claim 26, wherein the amount of 2-methyloxolane administered to the oviparous animal is comprised between 0.1 mg/kg of the oviparous animal per day and 200 mg/kg of the oviparous animal per day.

28. The method according to claim 26, wherein the dietary supplement is in the form of a solution, a powder, a capsule, an extrudate, a tablet or is a biological substrate cake.

29. The method according to claim 26, wherein the dietary supplement is a biological substrate cake.

30. The method according to claim 29, wherein the biological substrate cake is in the form of a cake or extrudate flour.

31. The method according to claim 29, wherein the biological substrate may be a plant selected from among almond, amaranth, peanut, argan, sea buckthorn, cashew tree, avocado, oats, borage, safflower, camelina, carrot, cocao, cashew, canola, hemp, rapeseed, copra, marrow, cotton, croton, rosehip, fig, prickly pear, pomegranate, hop, illipe, jojoba, jasmine, shea tree, lavender, lavandin, flax, lupin bean, corn, hazel tree, walnut, coconut palm, poppy, olive, evening-primrose, orange tree, oil palm, paprika, pecan, pistachio, pepper, castor, rice, rose, cedar rose, musk rose, sesame, soybean, marigold, sunflower, Calophyllum inophyllum, madhuca, Queensland nut, raspberry, blackcurrant, melon, grape, tomato, baobab, babassu, cranberry, chia, pumpkin, mustard, neem, Nigella Sativa, niger, poppy, Perilla, Plukenetia volubili winter squash, annatto, Taramira, apricot, plum, peach, wheat, tuberose, ylang-ylang, geranium, patchouli, petitgrain (citrus), anise, caraway, juniper, bergamot, mandarin orange, grapefruit, lemon, nutmeg tree, celery, angelica, cardamom, cumaru, vanilla, vetiver, iris, sandalwood, guaiacum, tarragon, citronella, sage, mint, Pine tree, rosemary, fir tree, thyme, galbanum, elemi, benzoin, myrrh, olibanum tree and mixtures thereof.

32. A dietary supplement for feeding an oviparous animal, said dietary supplement comprising 2-methyloxolane.
